(19) United States
(12) Patent Application Publication
(10) Pub. No.: US 2023/0211028 A1
(43) Pub. Date: Jul. 6, 2023

(54) AIR PURIFICATION APPARATUS AND METHODS OF AIR PURIFICATION AND TREATMENT USING IONIZATION

(71) Applicant: TOP PRODUCT INNOVATIONS, INC., Indian Trail, NC (US)

(72) Inventors: Robert MILLER, Indian Trail, NC (US); John NEWPORT, Indian Trail, NC (US); Alan ROSENBERG, Indian Trail, NC (US)

(21) Appl. No.: 17/762,509

(22) PCT Filed: Sep. 17, 2020
(86) PCT No.: PCT/US2020/051211

§ 371 Date: Mar. 22, 2022

Related U.S. Application Data
(60) Provisional application No. 62/901,518, filed on Sep. 17, 2019.

(51) Int. Cl. A61L 2/14; A61L 2/26; B03C 3/41; B03C 3/017; B03C 3/38; B03C 3/82; B01D 53/32; H01T 23/00

(52) U.S. Cl. CPC A61L 2/14; A61L 2/26; B03C 3/41; B03C 3/0175; B03C 3/38; B03C 3/82; B01D 53/323; H01T 23/00; A61L 2202/11; A61L 2202/25; B01D 2259/818; B01D 2257/90; B01D 2257/91

(57) ABSTRACT

An air purification apparatus and methods of air purification and treatment using ionization is disclosed. In some embodiments, an ion generator apparatus comprises a modular electrode and a housing connected to the modular electrode. The modular electrode includes an elongated conduit body comprising a power receiving end and a conduit connecting end opposite the power receiving end; and a plurality of ion generating elements occupying different radial positions around a perimeter of the conduit body, the ion generating elements generating negative ions or positive ions in response to an applied alternating current. A first electrical connector on the power receiving end is connectable to a second electrical connector on the conduit connecting end of another conduit body, such that a plurality of conduit bodies can be connected together in a series. The housing is connected to a power receiving end of one of the conduit bodies of the modular electrode.

### TECHNICAL FIELD

The presently disclosed subject matter relates generally to materials, devices, and methods of air purification and treatment and more particularly to air purification apparatus and methods of air purification and treatment using ionization.

### BACKGROUND

Cold Plasma Generators (CPG) produce an electric field filled with highly charged ions. This electrified field is known as a plasma field. Ions created within the plasma field can act as a natural scrubbing agent for air passing through the field. Particulates in the air, such as dust, mold, pollen, bacteria, viruses, and other harmful pathogens, pass through the plasma field, and the highly charged ions within the plasma field surround these particulates and breakdown their molecular structure. Pathogens and airborne viruses are destroyed as the ions bind them and change their molecular structures, often by robbing them of vital hydrogen molecules. In the case of odor-causing molecules, these molecules are often decomposed into atmospheric gases when passed through the plasma field through oxidation. In some cases, like particles agglomerate together when in the plasma field, making them larger and then easier to capture with an air filter.

The general concept of using ionization to generate positive and negative ions in air purification systems dates back to the 1960s with different ion generators having been developed over the years. However, many of these conventional ion generators are a “one size fits all” design, where the air purification systems have to be designed around the ion generator. However, this is not always possible, so the end results can be less than optimal. Additionally, these conventional ion generators use multiple electrodes, which increases the complexity, efficiency, and power consumption of the ion generator. Consequently, there is a need for improved ion generators that are relatively simple, more efficient, and use less energy.

### SUMMARY

In one aspect, a modular electrode for an ion generator is provided. As described further herein, the modular electrode can generate positive and negative ions from a single electrode architecture, thereby simplifying the design of an air purification apparatus and associated systems. The modular electrode can also couple with one or more additional modular electrodes to provide an ion-generating electrode of any desired length. Briefly, a modular electrode for an ion generator apparatus comprises an elongated conduit body comprising a power receiving end, and a conduit connecting end opposite the power receiving end, and a plurality of ion generating elements occupying different radial positions around a perimeter of the conduit body, the ion generating elements generating negative ions or positive ions.

In some embodiments, the power receiving end of the modular electrode comprises a first electrical connector, and a conduit connecting end comprises a second electrical connector having a shape corresponding to the first electrical connector. The first electrical connector of the power receiving end in some instances is connectable to the second electrical connector on the conduit connecting end of another conduit body. Thus, in some embodiments, a plurality of conduit bodies may be connected together in a series by connecting the first electrical connector of one conduit body to the second electrical connector of another conduit body. Each of the connected conduit bodies can have a same or a different length than the other conduit bodies. Furthermore, each conduit body can be made of an insulating material.

In some embodiments, a modular electrode described herein comprises an endcap connected to the conduit connecting end. Moreover, in some embodiments, a modular electrode can comprise an endcap having a first electrical connector connected to the second connector of the last conduit body in a series of connected conduit bodies.

A conduit body described herein can also comprise a plurality of ridges, wherein each ridge is positioned between adjacent ion generating elements.

Ion generating elements occupy different radial positions around the perimeter of the conduit body. In some embodiments, for example, the perimeter of the conduit body comprises a first surface and an oppositely facing second surface extending parallel along a length of the conduit body. A plurality of ion generating elements can be positioned along the length of the first surface and the oppositely facing second surface of the conduit body. Accordingly, the ion generating elements exhibit a radial spacing of 180 degrees along the perimeter of the conduit body. Ion generating elements can exhibit any desired radial spacing along the perimeter of the conduit body. The plurality of ion generating elements can generate positive ions and negative ions in a fluid stream, such as air, when energized with alternating current. Generation of the positive and negative ions by the elements can be dependent on the positive and negative cycles of the alternating current.

In another aspect, an ion generator apparatus comprises a modular electrode described herein, and a housing connected to the power receiving end of the conduit body of the modular electrode. Electronic circuitry can be positioned in the housing, and can be configured to deliver alternating electric current to the conduit body and the ion generating elements.

In some embodiments, a housing can be made of a translucent material, and optionally one or more light-emitting diode (LED) lights can be positioned inside the housing, the one or more LED lights illuminating the housing when the ion generator apparatus is operating.

In some cases, a housing described herein can comprise a fastener. The fastener can be a securing flange in some cases, and the securing flange can be positioned on the same side of the housing where the conduit body is connected. In some instances, the securing flange provides a seal into a conduit or plenum into which a modular electrode portion of the ion generator apparatus has been inserted. Additionally, in some embodiments the securing flange comprises tabs that secure the housing to the conduit or plenum.

In another aspect, a method of purifying air comprises providing an ion generator apparatus described herein; connecting two or more of the conduit bodies together; and positioning the connected conduit bodies in a source of air to be purified. The method can further comprise in some instances generating negative ions or positive ions with the plurality of ion generating elements positioned along the length of the conduit body. Furthermore, the method can further comprise passing air to be purified over the ion generating elements.

In some embodiments, a method described herein can comprise fastening the housing of the ion generator apparatus to a heating, ventilation, and air conditioning (HVAC) conduit or plenum.

### BRIEF DESCRIPTION OF DRAWINGS

Having thus described the presently disclosed subject matter in general terms, reference will now be made to the accompanying drawings, which are not necessarily drawn to scale, and wherein:

FIG. 1, FIG. 2, FIG. 3, and FIG. 4 illustrate various perspective views of an example of a modular electrode of the presently disclosed air purification apparatus that uses ionization;

FIG. 5A and FIG. 5B illustrate perspective views of an example of two modular electrodes being connected together;

FIG. 6 illustrates a schematic diagram of an example of an electronic circuit the presently disclosed air purification apparatus that uses ionization;

FIG. 7 and FIG. 8 illustrate perspective views of an example of an ion generator apparatus of the presently disclosed air purification apparatus;

FIG. 9 illustrates a side view of the ion generator apparatus shown in FIG. 7 and FIG. 8;

FIG. 10 illustrates a side view of the ion generator apparatus shown in FIG. 7 and FIG. 8 connected to an HVAC conduit;

FIG. 11 illustrates an example of a flow diagram of a method of purifying air using the presently disclosed air purification apparatus that uses ionization;

FIG. 12A and FIG. 12B illustrate a side view and an exploded view, respectively, of another example of an ion generator apparatus of the presently disclosed air purification apparatus; and

FIG. 13A, FIG. 13B, FIG. 13C, and FIG. 13D illustrate various views of an example of a modular electrode of the ion generator apparatus shown in FIG. 12A and FIG. 12B.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides an air purification apparatus and methods of air purification and treatment using ionization. In some embodiments, an ion generator apparatus is provided that can produce an electric field filled with highly charged ions, also known as a plasma field. In some embodiments, when air is contacted with the ion generator, O2−(H2O)x negative ions and H+P(H2O)y positive ions are generated from molecules of water contained as moisture in the air, where x and y are positive integers. These positive and negative ions react with particulates in the air, such as dust, mold, and pollen, and induce agglomeration, which creates larger particles that are more easily capture by an air filter than the smaller original particles.

Additionally, these positive and negative ions can react with and kill bacteria, viruses, and other pathogens in the air. For example, when O2−(H2O)x negative ions and H+P(H2O)y positive ions attach to the surfaces of airborne pathogens, radical hydroxyl (OH·) and hydrogen peroxide (H2O2) are generated on the surface of the pathogens, and critical hydrogen atoms are extracted from the pathogen surface by the radical hydroxyl and hydrogen peroxide, thereby killing them.

Moreover, the O2−(H2O)x negative ions and H+P(H2O)y positive ions react with various odor-causing small molecules in the air, and chemically oxidize these odor-causing small molecules, thereby effectively deodorizing the air.

### Section I.—Modular Electrode

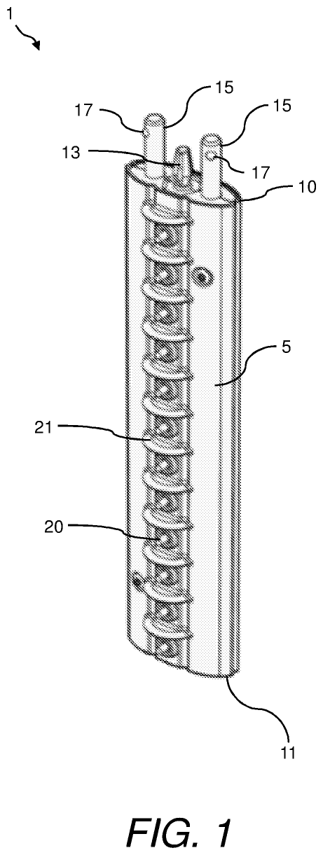

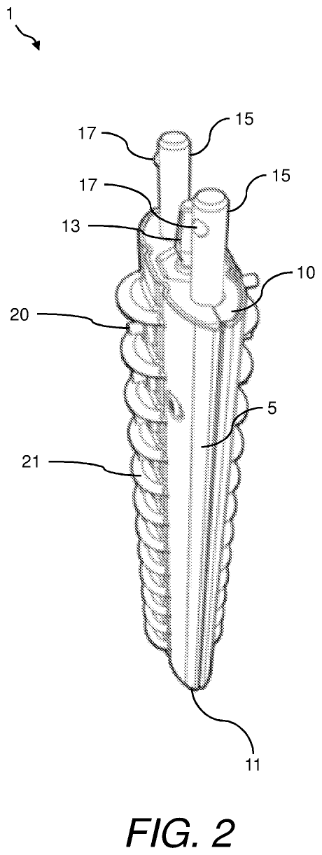

In one aspect, a modular electrode for an ion generator is described herein. For example, FIG. 1, FIG. 2, FIG. 3, and FIG. 4 show various perspective views of an exemplary embodiment of a modular electrode 1. In some embodiments, the modular electrode 1 described herein comprises an elongated conduit body 5 and a plurality of ion generating elements 20. Modular electrode 1 can also be referred to as a “blade” or a “Stinger”.

A conduit body, such as conduit body 5, may be made of any material not inconsistent with the objectives of this disclosure. In some embodiments, conduit body 5 may be made of an insulating material, such as a plastic or resin. Exemplary plastics include polyolefins, such as polypropylene or polyethylene, vinylic-based polymers, such as PVC and ABS, acrylate-based polymers, and the like. Conduit body 5 may be made in any manner known to those skilled in the art, such as through injection molding. Alternatively, conduit body 5 may be fabricated via one or more additive manufacturing technique, such as binder jetting.

The conduit body 5 described herein may have any shape that allows air to pass by and contact a plasma field created by the ion generating elements, such as an aerodynamic shape. In some embodiments, conduit body 5 has a blade-like shape, as shown in FIG. 1, FIG. 2, FIG. 3, and FIG. 4. In other cases, conduit body 5 may have a cross-sectional shape that is oval, circular, squared, rectangular, triangular, pentagonal, hexagonal, and the like. Further, conduit body 5 may comprise a first surface 18A and an oppositely facing second surface 18B extending parallel along a length of conduit body 5, as shown in FIG. 4.

In some embodiments, conduit body 5 comprises a power receiving end 10 and a conduit connecting end 11 opposite the power receiving end 10, as illustrated in FIG. 1, FIG. 2, FIG. 3, and FIG. 4. The power receiving end 10 can comprise a first electrical connector and the conduit connecting end 11 can comprise a second electrical connector having a shape or recess corresponding to the first electrical connector. For example, in some embodiments, the power receiving end 10 can be a male-style plug and the conduit connecting end 11 can be a female-style socket into which the power receiving end 10 plug can be inserted. However, the plug and socket style connector is exemplary only, and other types of connectors are also contemplated.

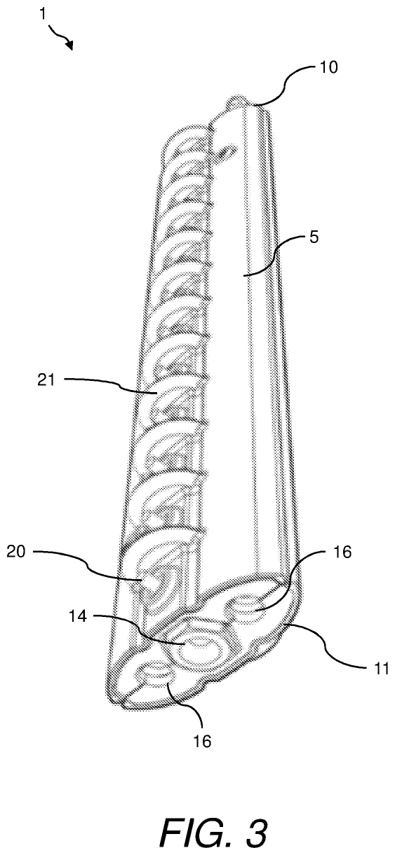

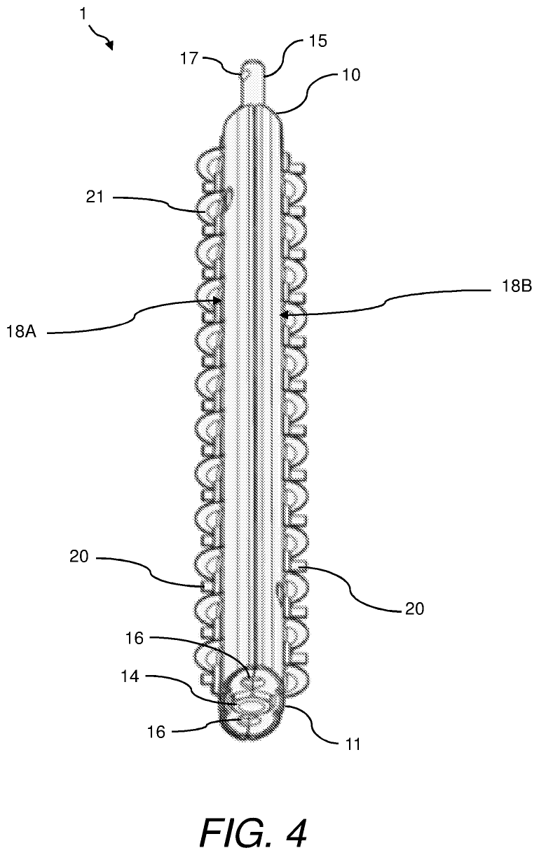

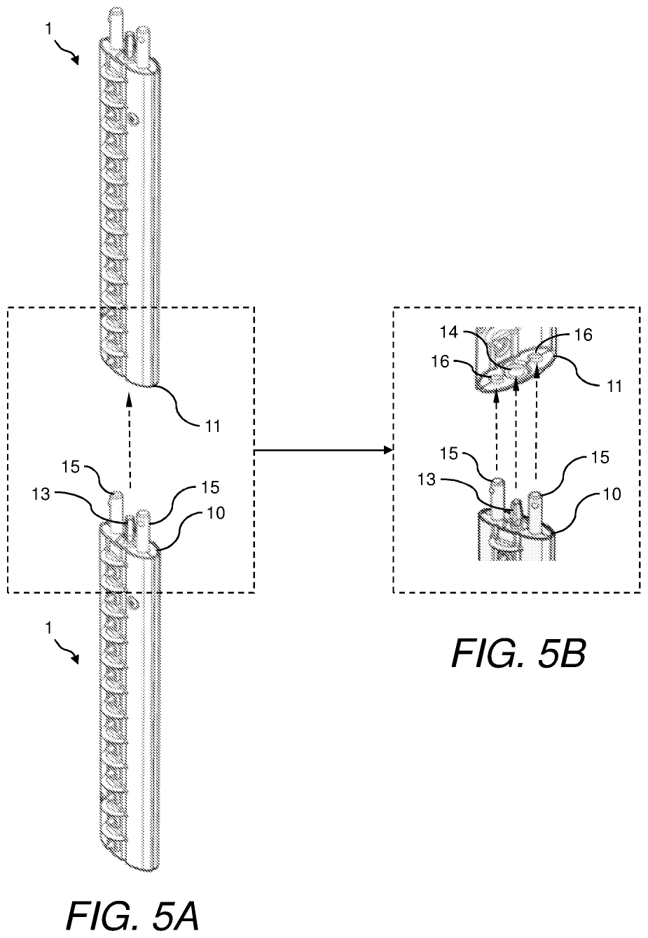

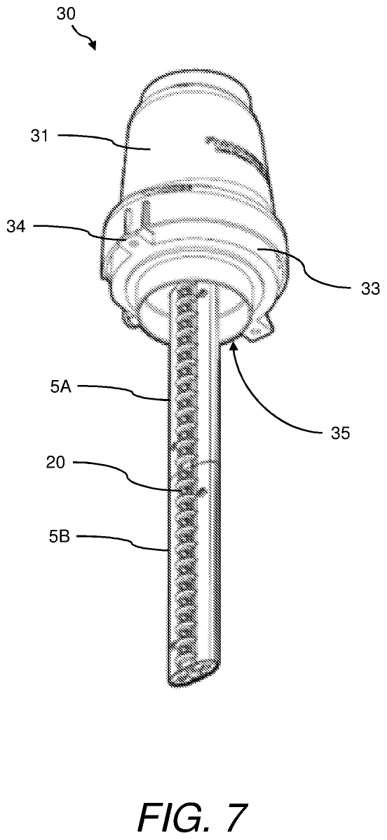

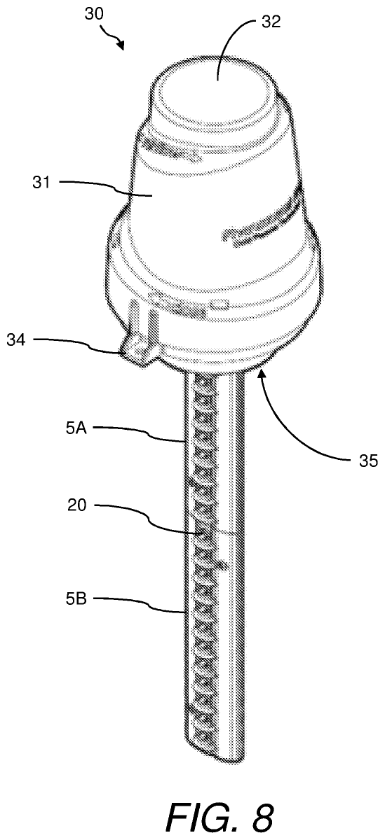

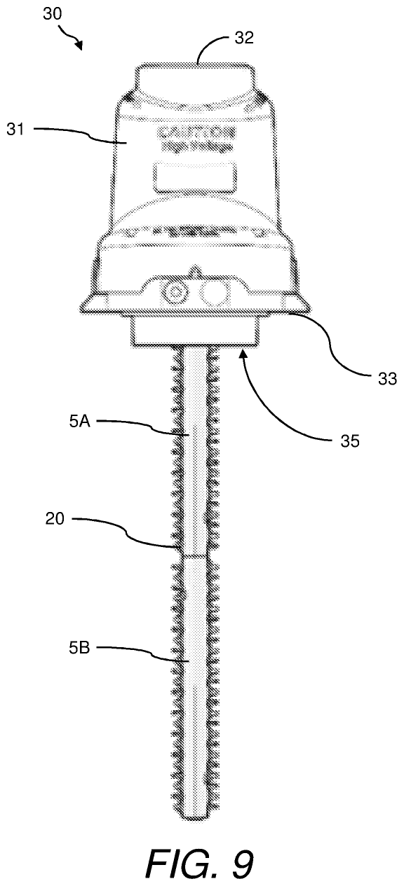

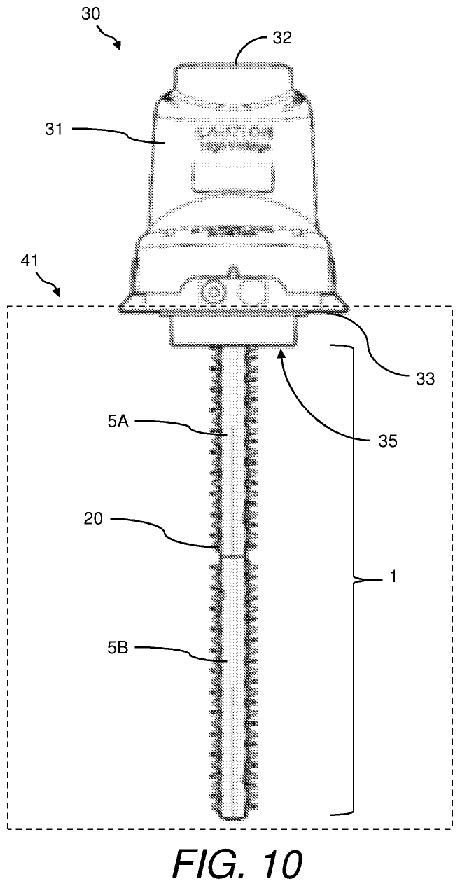

FIG. 5A and FIG. 5B show a plug and socket style of connection where the first electrical connector of the power receiving end 10 comprises one or more electrically conductive blades, prongs, or wires 13, and the second electrical connecter of the conduit connecting end 11 comprises one or more electrically conductive sockets 14. Multiple conduit bodies 5 described herein can therefore be connected together by connecting the first electrical connector of a power receiving end 10 of one conduit body 5 with a second electrical connector on a conduit connecting end 11 of another conduit body 5, as illustrated for example in FIG. 7, FIG. 8, FIG. 9, and FIG. 10. In some embodiments, a plurality of conduit bodies 5 can be connected together in a series by connecting the first electrical connector of one conduit body 5 to the second electrical connector of another conduit body 5.

The conduit body 5 described herein can comprise a fastener assembly that fastens one conduit body 5 to another conduit body 5. In some instances, the fastener assembly comprises a second plug and socket assembly that is different from a first electrical connector and a second electrical connector described herein. Generally, components comprising the fastener assembly are made of a non-conductive material, such as the same material comprising the conduit body 5 itself. In some embodiments, components of the fastener assembly are integrally formed on a conduit body. In some cases, conduit body 5 can comprise a fastener assembly having a plug on one of the power receiving end 10 or conduit connecting end 11, and a complimentarily shaped socket positioned on the other of the power receiving end 10 or conduit connecting end 11.

FIG. 1 through FIG. 5B illustrate an exemplary plug and socket-based fastener assembly of modular electrode 1, where two fastener prongs 15 extend outward from a power receiving end 10 of a conduit body 5. Two complimentarily shaped fastener prong receiving sockets 16 are positioned on a conduit connecting end 11 of a conduit body 5.

In one example, power receiving end 10 includes one electrically conductive blade, prong, or wire 13 flanked by two fastener prongs 15 (see FIG. 5B). In complementary fashion, conduit connecting end 11 includes one electrically conductive socket 14 flanked by two receiving sockets 16 (see FIG. 5B). The fastener prongs 15 can be inserted into the receiving sockets 16, as illustrated in FIG. 5A and FIG. 5B, to connect two conduit bodies 5 together. The fastener prongs 15 can be held in the receiving sockets 16 through a friction fit, or, in some embodiments, the fastener prongs 15 can include a locking protrusion 17 and the receiving sockets 16 can correspondingly comprise a locking protrusion receiving detent or catch (not shown). In these embodiments, two conduit bodies 5 are held together when the two locking protrusions 17 of the two fastener prongs 15 of the first conduit body 5 are engaged with the two locking protrusion receiving detents or catches of the two receiving sockets 16 of the second conduit body 5.

In other embodiments, a fastener assembly of modular electrode 1 can comprise mechanisms other than a plug and socket. For instance, in some embodiments, a fastener assembly can comprise a latch and catch assembly (not shown), where two resilient latching arms are positioned on opposite sides of one of a power receiving end 10 and a conduit connecting end 11 of conduit body 5, and two complimentary shaped latch receiving catches are positioned on the other of the power receiving end 10 and conduit connecting end 11 of conduit body 5. Two or more conduit bodies 5 can be connected together by contacting the power receiving end 10 of one conduit body 5 with the conduit connecting end 11 of another conduit body 5 such that the resilient latching arms of the one conduit body 5 engage and latch to the latch receiving catches of the other conduit body 5.

The conduit body 5 of modular electrode 1 can have any length not inconsistent with the objectives of this disclosure. In instances where a plurality of conduit bodies 5 are connected together in a series, each of the connected conduit bodies 5 can have the same or a different length from the other conduit bodies 5. Thus, in this manner, a length of a modular electrode 1 can be customized to fit any application by connecting any number of conduit bodies 5 together to form a modular electrode 1 having a desired length.

In some embodiments, a modular electrode 1 described herein can comprise an endcap. An endcap described herein can comprise a fastener assembly, a first electrical connector, or both a fastener assembly and a first electrical connector. The endcap can be connected to a conduit connecting end of a conduit body 5 described herein. In embodiments where a plurality of conduit bodies 5 are connected in series, the endcap can be connected to a second connector on a conduit connecting end of the last conduit body 5 in the series. The endcap can provide one or more advantages, such as preventing debris from entering second electrical connector sockets and/or fastener assembly sockets on a conduit connecting end of a terminating conduit body 5 of the modular electrode 1. Additionally, in some embodiments, a first electrical connector on the endcap can complete an electrical circuit positioned in the conduit body 5.

A conduit body described herein can also comprise a plurality of receiving spaces and a plurality of ridges. As shown for example in FIG. 1 through FIG. 4, a plurality of ridges 21 are positioned on a surface of the conduit body 5 between two different receiving spaces. As explained in more detail below, each receiving space has an ion generating element 20 positioned therein. Consequently, each receiving space can be identified as being positioned where each ion generating element 20 is shown in FIG. 1 through FIG. 4. The ridges 21 separate each ion generating element 20 from each other, thus isolating and preventing interaction between the ion generating elements 20.

A modular electrode 1 described herein comprises a plurality of ion generating elements 20. The ion generating elements 20 are radially spaced at different positions around a perimeter of conduit body 5. For example, as shown in FIG. 1 through FIG. 4, a plurality of ion generating elements 20 can occupy different radial positions around a perimeter of the conduit body 5. Particularly shown in FIG. 4, a plurality of ion generating elements 20 can be positioned along the length of the first surface 18A and the oppositely facing second surface 18B of the conduit body 5. However, this configuration is exemplary, and in other embodiments, the ion generating elements 20 can be radial spaced from each other at different positions and patterns. For example, while FIG. 1 through FIG. 4 show embodiments where the ion generating elements 20 occupy different radial positions along the length of the conduit body 5 in a straight line, in other embodiments, the ion generating elements 20 can be staggered along a length of each side of conduit body 5. In some cases, the ion generating elements 20 can be radially spaced in a spiral extending along a length of conduit body 5. In some embodiments, the ion generating elements 20 protrude outward from conduit body 5 to insert the ion generating element 20 into an airstream.

Ion generating elements 20 are capable of generating negative ions or positive ions when energized. In some embodiments, each ion generating element 20 can generate negative ions or positive ions when energized with alternating current. The alternating current can be any input voltage not inconsistent with the objectives of this disclosure. For example, the alternating current can be 12V, 120V, or 208-240V. In some less preferred embodiments, direct current can be used to energize the ion generating elements 20, such as 12 v or 24 v direct current.

Ion generating elements 20 described herein can be made of any electrically conductive material capable of generating a plasma field having negative ions and/or positive ions in an air stream. Exemplary materials include steel (stainless or non-stainless), copper, aluminum, tungsten, conductive carbon fiber, carbon-doped polyolefins such as polypropylene, and other conductive metals and materials. Additionally, ion generating elements 20 can have any desired morphology and/or architecture for generating ions in an air stream. Ion generating elements 20 may exhibit, for example, needle or needle-like architectures. In some embodiments, ion generating elements 20 are bundles of needles.

In some embodiments, the plurality of ion generating elements 20 produce negative ions and positive ions in equal quantities. In other embodiments, the plurality of ion generating elements 20 produce negative and positive ions in unequal quantities, such as more negative ions than positive ions in some cases, or more positive ions than negative ions in other cases. Moreover, in some embodiments, the plurality of ion generating elements 20 can produce only negative ions, or only positive ions. In some cases, a ratio of negative ions to positive ions can be controlled by using different pulse waveforms of alternative current.

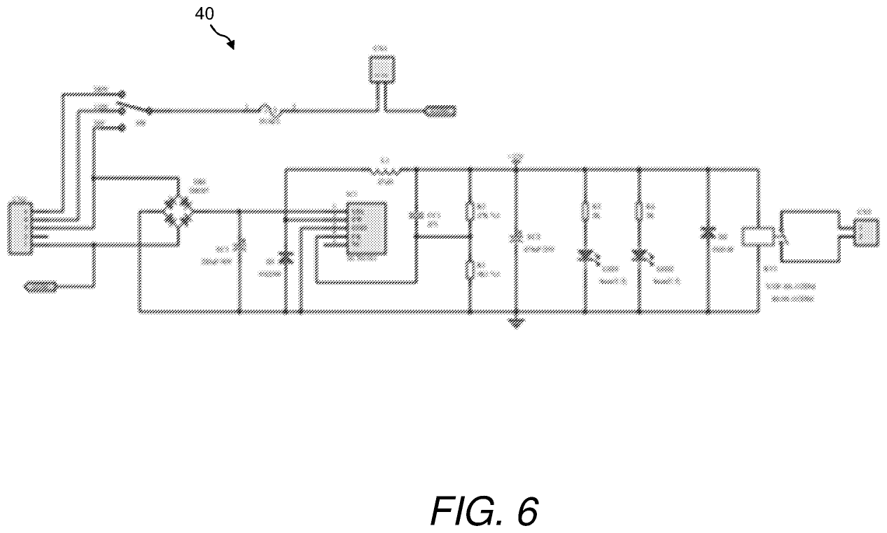

A modular electrode 1 described herein can further comprise wiring routed through conduit body 5 connecting the plurality of ion generating elements 20 to an external power source. FIG. 6 shows a schematic diagram of an example of an electronic circuitry 40 of a modular electrode 1 connected to a housing described in more detail in Section II hereinbelow.

One advantage of the presently disclosed modular electrode 1 over other ion generating electrodes, is that little to no ozone is produced during the generation of the negative ions and/or positive ions.

### Section II.—Ion Generator Apparatus

In another aspect, the presently disclosed ion generator apparatus is described herein. In some embodiments, an ion generator apparatus comprises a modular electrode described in Section I above, and a housing.

An exemplary embodiment of an ion generator apparatus 30 is shown in FIG. 7, FIG. 8, and FIG. 9, the ion generator apparatus 30 comprising a housing 31 connected to a power receiving end 10 of the conduit body 5 of a modular electrode 1. Particularly shown in FIG. 7, FIG. 8, and FIG. 9 is a modular electrode 1 having two conduit bodies 5 (e.g., conduit bodies 5A, 5B) connected together in a manner described in Section I herein, although the ion generator apparatus 30 is not limited to two conduit bodies 5. In other embodiments, the ion generator apparatus 30 can only have one conduit body 5, or, in other embodiments, the ion generator apparatus can comprise a plurality of conduit bodies 5, such as 3, 4, 5, 6, 7, 8, 9, 10, or more.

In some embodiments, a removable cap 32 is secured to a side of the housing 31, such as on a top side. The removable cap 32 can be secured using any mechanism not inconsistent with the objectives of this disclosure, such as a threaded or latching mechanism. In one particular embodiment, the removable cap 32 can be secured to the housing 31 using a turn and lock mechanism where the removable cap 32 is inserted into a cap receiving opening in the housing 31, and the removable cap 32 is turned slightly to engage a locking mechanism in the removable cap 32 with a corresponding feature in the cap receiving opening in the housing 31. An optional gasket (not shown) can be positioned between the cap 32 and the housing 31 to provide a waterproof seal.

The housing 31 can made of the same material or a different material as the conduit body 5. In some embodiments, the housing 31 can be made from a plastic or a resin, such as a polyolefin, polyvinyl, polyacrylate, or any other suitable material not inconsistent with the objectives of this disclosure.

In some embodiments, the housing 31 comprises a space therein (not shown) for receiving certain electronics (e.g., electronic circuitry 40 shown in FIG. 6), where various electrical components are positioned. As shown in FIG. 6, the electronic circuitry 40 can be configured to deliver power to one or more conduit bodies 5 and the ion generating elements 20. As described for example in Section I, the electronic circuitry 40 can be configured to deliver alternating current of 12V, 120V, or 208-240V to the ion generating elements 20.

In some embodiments, the housing 31 is translucent. Optional light emitting diodes (LEDs) can be positioned in the housing as part of electronic circuitry 40, and the LEDs can illuminate the housing 31 when turned on. FIG. 6 shows an exemplary schematic diagram showing one embodiment comprising LEDs. In some embodiments, the housing 31 is opaque and the removable cap 32 is made of a translucent material. In this embodiment, when the LEDs are illuminated, the removable cap 32 shows the illumination. In some cases, different colored LEDs are positioned in the housing 31, and each color indicates a status of the ion generator apparatus 30. For example, a red color could indicate that the ion generator apparatus 30 is not currently operating, and a green color could indicate that the ion generator apparatus 30 is currently operating. However, this example is merely exemplary and any combination of colors can be used to indicate any particular operation.

A housing 31 described herein can comprise a fastening assembly in some cases. For example, as shown in FIG. 7, FIG. 8, and FIG. 9, a fastening assembly can comprise a securing flange 33 positioned on the same side of the housing 31 where the conduit body 5 is connected. Various connecting tabs 34 can be positioned around a perimeter of the securing flange 33, and the connecting tabs 34 can comprise fastener receiving holes where screws, bolts, rivets, or other fasteners can be inserted and connected to a conduit or plenum to secure the ion generator apparatus 30 to a surface thereof.

As shown for example in FIG. 7, FIG. 8, and FIG. 9, a power receiving end 10 of circuit body 5A is connected to securing end 35 of housing 31. Although not expressly shown in FIG. 7, FIG. 8, and FIG. 9, the securing end 35 of housing 31 can comprise a second electrical connector identical in design to the second electrical connector on the conduit connecting end 11 of conduit body 5A. Thus, conduit body 5A of a modular electrode 1 can be connected to the housing 31 in the same manner as two conduit bodies 5 are connected together as described in Section I. Consequently, if a conduit body 5 is damaged or needs to be replaced in an ion generator apparatus 30 described herein, the conduit body 5 can be unplugged from the housing and a new conduit body 5 can be plugged in.

Moreover, one or more conduit bodies 5 of a modular electrode 1 can be connected together to form a modular electrode 1 of any length, such as the conduit bodies 5A and 5B shown in FIG. 7, FIG. 8, and FIG. 9. This allows flexibility in providing an ion generator apparatus 30 that meets the needs of a variety of different needs or applications by permitting a user to employ an exact length of modular electrode 1 needed for a particular application.

FIG. 10 shows an exemplary embodiment of an ion generator apparatus 30 installed in an HVAC conduit 41, where the HVAC conduit 41 is shown as a cross-sectional box. As shown, modular electrode 1 has been inserted into an opening in the HVAC conduit 41, such that the modular electrode 1 is positioned in an airflow of air within the HVAC conduit 41. The housing 31 is positioned on an outer surface of the HVAC conduit 41, and the securing flange 33 rests on the outer surface of the HVAC conduit 41 and forms an airtight seal. Again, while FIG. 10 depicts an ion generator apparatus 30 having a modular electrode 1 with two conduit bodies 5A, 5B connected together, the ion generator apparatus 30 can comprise a modular electrode 1 having any number of conduit bodies 5 connected together as needed for a particular application.

### Section III.—Method of Purifying Air

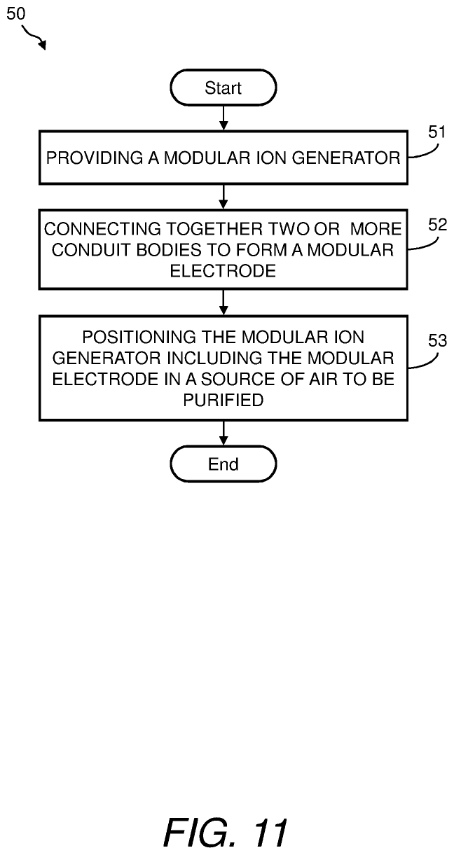

In another aspect, FIG. 11 shows an example of a method 50 of purifying air with an ion generator apparatus (e.g., ion generator apparatus 30). Method 50 may include, but is not limited to, the following steps.

At a step 51, a modular ion generator apparatus is provided. In one example, ion generator apparatus 30 as described in FIG. 7 through FIG. 10 in Section II that includes one or more modular electrodes 1 as described in FIG. 1 through FIG. 5B in Section I is provided.

At a step 52, two or more of the conduit bodies are connecting together to form a modular electrode of a desired length. In one example, two or more of the conduit bodies 5 are connecting together to form a modular electrode 1 of a desired length, as described in FIG. 1 through FIG. 10.

At a step 53, the ion generator apparatus including the modular electrode is positioned in a source of air to be purified. For example, ion generator apparatus 30 including the modular electrode 1 formed by the connected conduit bodies 5 is positioned in a source of air to be purified.

In some embodiments, the step 53 of method 50 of positioning the connected conduit bodies 5 comprises fastening the housing 31 of the ion generator apparatus 30 to an HVAC conduit 41 or plenum, as shown in FIG. 10.

In some embodiments, the method 50 described herein can further comprise generating negative ions or positive ions with the plurality of ion generating elements positioned along the length of the conduit body 5. Moreover, the method 50 described herein can further comprise passing air to be purified over the ion generating elements 20. As air is passed over, the ion generating elements 20, particles, molecules, and pathogens in the air pass through the plasma field being generated by the ion generating elements 20, and react with the negative and/or positive ions. The particles agglomerate together in some instances to form larger particles that are then more easily captured by air filters, or the particles become too large to be airborne, and precipitate out of the air. Odor causing molecules can become oxidized, eliminating or reducing their odor causing abilities. The negative and positive ions react with the surfaces of the airborne pathogens, extracting critical hydrogen atoms or oxidizing critical cellular or viral components, killing the pathogens.

### Section IV.—Modular Electrode

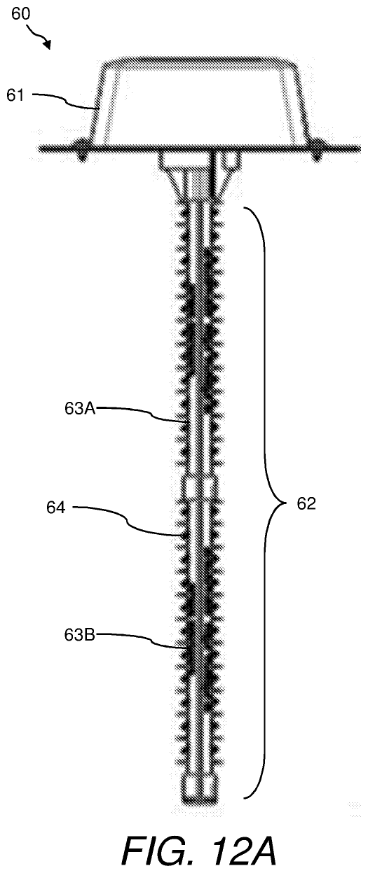

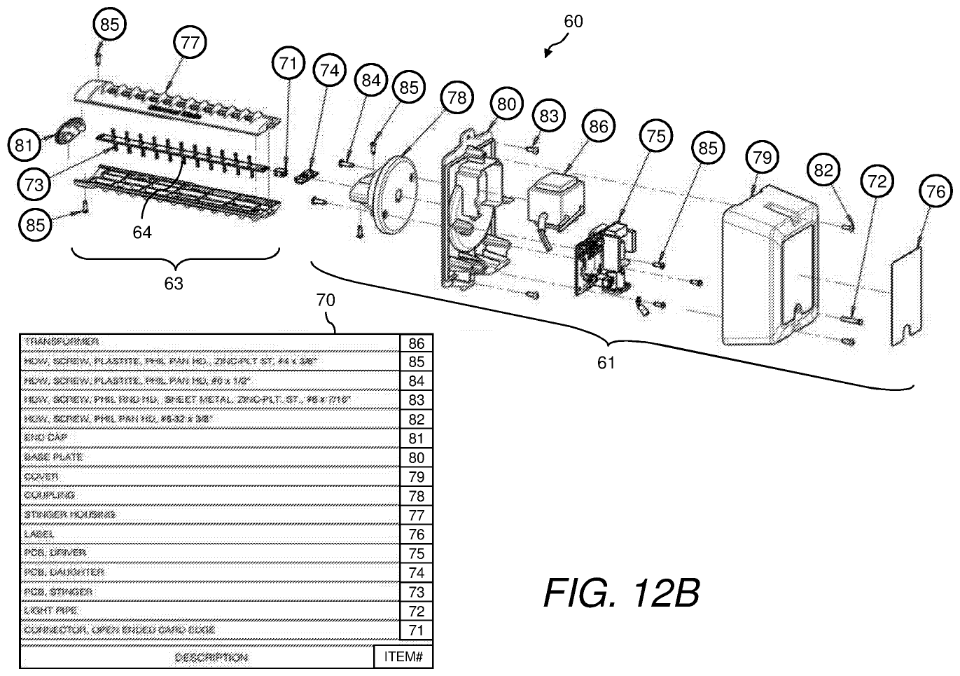

Referring now to FIG. 12A and FIG. 12B is a side view and an exploded view, respectively, of another example of an ion generator apparatus 60 of the presently disclosed air purification apparatus. In one example, ion generator apparatus 60 includes a housing 61 coupled to a modular electrode 62 formed of an arrangement of conduit bodies 63 (e.g., conduit bodies 63A, 63B). Further, each of the conduit bodies 63 holds an arrangement of ion generating elements 64.

FIG. 12B shows the various components of ion generator apparatus 60 including housing 61 and modular electrode 62. With reference to parts list 70, a conduit body 63 of ion generator apparatus 60 may include, but is not limited to, a Stinger PCB 73 holding the arrangement of ion generating elements 64, a Stinger housing 77, a connector 71, and an endcap 81. The components held in housing 61 of ion generator apparatus 60 may include, but are not limited to, a light pipe 72, a PCB daughter board 74, a driver PCB 75, a label 76, a coupling 78, a cover 79, a baseplate 80, and a transformer 86. Further, ion generator apparatus 60 includes multiple screws 82, 83, 84, 85.

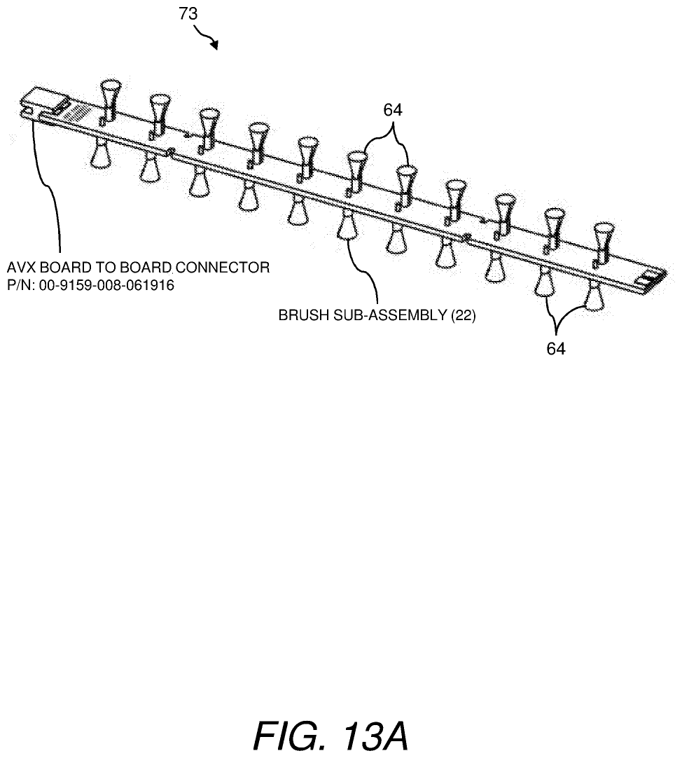

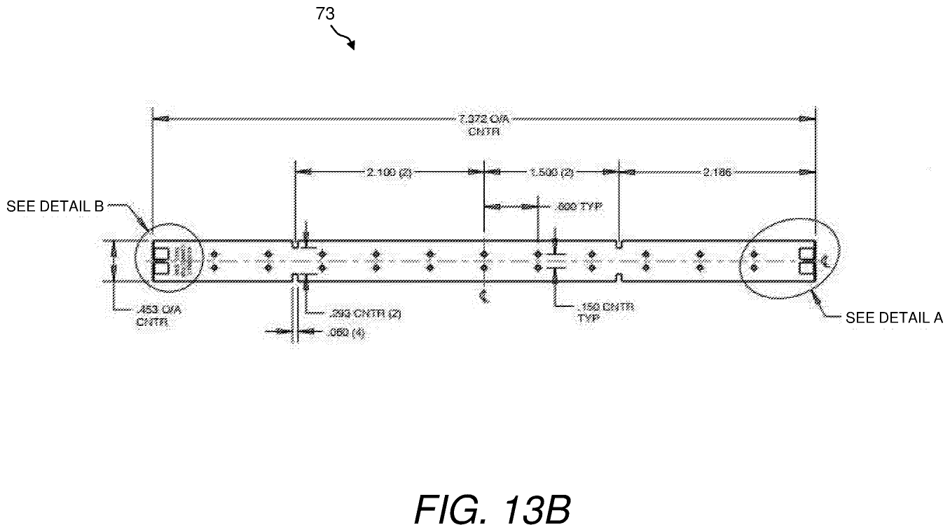

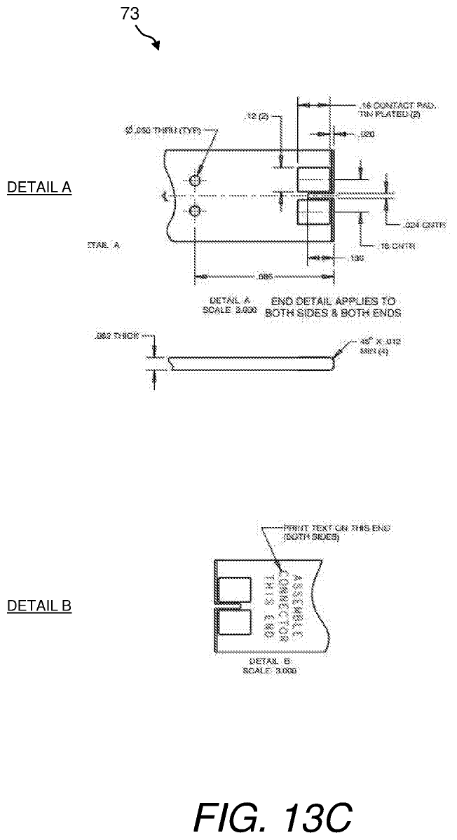

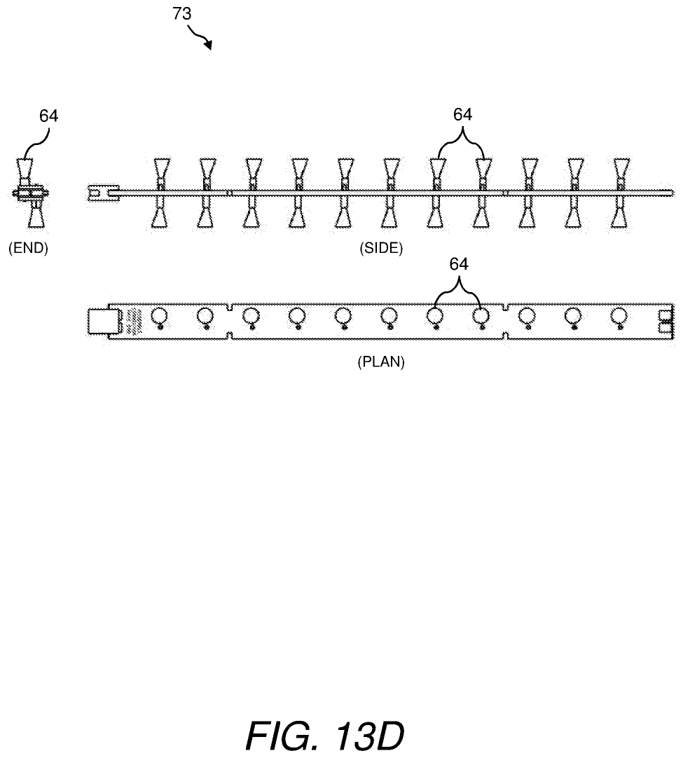

Referring now to FIG. 13A, FIG. 13B, FIG. 13C, and FIG. 13D is various views of an example of modular electrode 62 of ion generator apparatus 60 shown in FIG. 12A and FIG. 12B. For example, FIG. 13A shows a perspective view of Stinger PCB 73 and FIG. 13B shows a plan view of Stinger PCB 73 of modular electrode 62. FIG. 13C shows a Detail A and a Detail B of FIG. 13B. FIG. 13D shows a plan view, a side view, and an end view of Stinger PCB 73 of modular electrode 62.

Referring now again to FIG. 12A through FIG. 13D, the design of ion generator apparatus 60 generally include reinforcing features to provide good rigidity. Further, the design of ion generator apparatus 60 provides a high frequency air cleaner blade that may include, but is not limited to, the following physical attributes:
(1) Conduit body 63 consists of two halves of the same plastic part (e.g., Stinger housing 77) assembled around a single circuit board (e.g., Stinger PCB 73) consisting of twenty-two (22) carbon fiber brushes (e.g., ion generating elements 64) and a one board-to-board connector (e.g., connector 71);
(2) The circuit board (e.g., Stinger PCB 73) and connector combination may be snapped into the two identical plastic halves (Stinger housing 77) allowing the carbon brushes (e.g., ion generating elements 64) to protrude the optimum distance for operation;
(3) Ion generator apparatus 60 is thin with a streamlined body that reduces obstruction and provides additional air flow over the carbon fiber brushes (e.g., ion generating elements 64);
(4) Ion generator apparatus 60 is designed without the use of heavy and expensive mechanical connectors that are difficult to assemble to the circuit board, and thereby increasing reliability and functionality and lowering the cost of the assembly;
(5) Ion generator apparatus 60 provides a male and female (hermaphroditic) connection that includes mating surfaces simultaneously. This connection is then secured by two screws per end;
(6) Ion generator apparatus 60 allows for multiple conduit bodies 63 to be assembled together with little flex;
(7) Ion generator apparatus 60 provides a high frequency unit that provides a compact footprint, ease of assembly, and high reliability; and
(8) Ion generator apparatus 60 provides a small, low profile unit that is capable of both integrated assembly as well as remote assembly by the use of a 3-foot high voltage cable.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

1. A modular electrode for an ion generator apparatus comprising:
an elongated conduit body including:
a power receiving end, and
a conduit connecting end opposite the power receiving end; and
a plurality of ion generating elements occupying different radial positions around a perimeter of the elongated conduit body, the plurality of ion generating elements generating negative ions or positive ions.

2. The modular electrode of claim 1, wherein
the power receiving end comprises an electrical connector, and
the conduit connecting end comprises an electrical connector having a shape corresponding to the electrical connector of the power receiving end.

3. The modular electrode of claim 1, wherein an electrical connector of the power receiving end is connected to an electrical connector of a conduit connecting end of another conduit body.

4. The modular electrode of claim 1, wherein the radial positions of the plural ion generating elements are separated by at least 90 degrees.

5. (canceled)

6. (canceled)

7. The modular electrode of claim 3, further comprising:
an endcap having an electrical connector connected to an electrical connector of the conduit connecting end or the electrical connector of the other conduit connecting end.

8. The modular electrode of claim 1, further comprising:
an endcap connected to the conduit connecting end.

9. The modular electrode of claim 1, further comprising:
a plurality of ridges, wherein each ridge is positioned between adjacent ion generating elements.

10. The modular electrode of claim 1, wherein the elongated conduit body comprises an insulating material.

11. (canceled)

12. The modular electrode of claim 1, wherein the plurality of ion generating elements are positioned along a length of a first surface and an oppositely facing second surface extending between the power receiving end and the conduit connecting end of the elongated conduit body.

13. The modular electrode of claim 1, wherein the ion generating elements comprise bundled needle-like architectures.

14. The modular electrode of claim 1, wherein the plurality of ion generating elements generate the positive ions and negative ions when energized with alternating current.

15. An ion generator apparatus comprising:
a modular electrode; comprising:
a conduit body including a power receiving end; and
ion generating elements occupying different radial positions around the conduit body; and
a housing connected to the power receiving end of the conduit body of the modular electrode.

16. The ion generator apparatus of claim 15, further comprising:
electronic circuitry positioned in the housing, the electronic circuitry configured to deliver power to the conduit body and the ion generating elements.

17. The ion generator apparatus of claim 15, wherein the housing comprises a fastening assembly.

18. The ion generator apparatus of claim 17, wherein the fastening assembly is a securing flange positioned on a side of the housing connected to the conduit body.

19. The ion generator apparatus of claim 17, wherein the housing is translucent.

20. The ion generator apparatus of claim 19, wherein the housing comprises one or more LED lights positioned inside the housing, the one or more LED lights illuminating the housing when the ion generator apparatus is operating.

21. A method of purifying air comprising:
positioning, in a source of air to be purified, an ion generator apparatus including a modular electrode with a conduit body and plural ion generating elements occupying different radial positions around the conduit body;
generating negative ions or positive ions via the plural ion generating elements; and
causing the generated negative or positive ions to interact with particles, molecules and/or pathogens in the source of air.

22. (canceled)

23. (canceled)

24. The method of claim 21, wherein the ion generator apparatus is fastened to an HVAC conduit or plenum.

25. The method of claim 21, wherein the radial positions of the plural ion generating elements are separated by at least 90 degrees.
